(19) United States
(12) Patent Application Publication
(10) Pub. No.: US 2018/0214070 A1
(43) Pub. Date: Aug. 2, 2018

(54) Nevigraph

(71) Applicant: Michael Darin McDaniel, Highland Village, TX (US)

(72) Inventor: Michael Darin McDaniel, Highland Village, TX (US)

(21) Appl. No.: 15/422,446

(22) Filed: Feb. 1, 2017

(51) Int. Cl. A61B 5/00; A61B 5/107; A61B 5/103

(52) U.S. Cl. CPC A61B 5/444; A61B 5/1034; A61B 5/1072

(57) ABSTRACT

The Nevigraph is a credit card sized (2.25″×3.25″) plastic card designed for use in dermatology offices and given to patients to monitor any mole changes over time between dermatologist visits. It includes a millimeter ruler, a range of 10 shades of brown/black with holes to place over the mole while comparing color, a place for the dermatologist to record the mole location, a space for the dermatologist to put their contact information, and a back side that includes descriptions and example pictures illustrating the ABCDEs of dermatology.

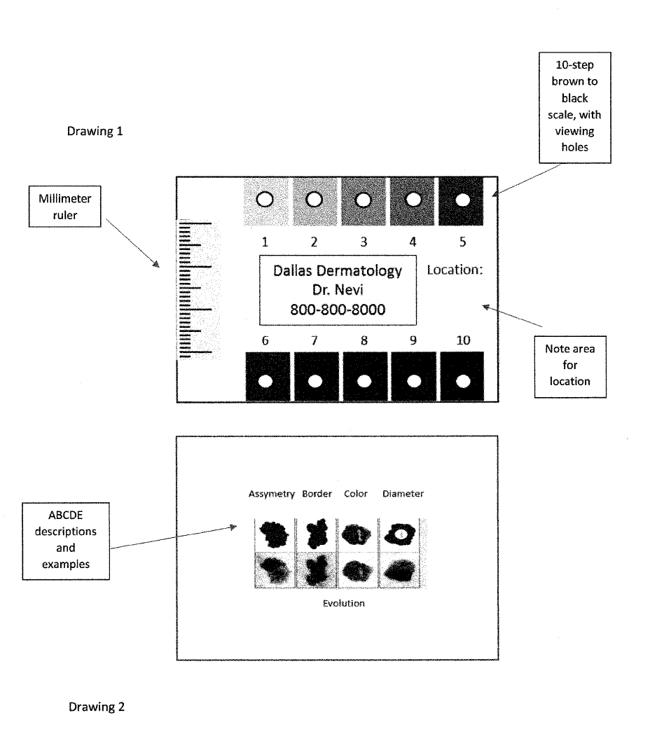

### CROSS REFERENCE TO RELATED APPLICATIONS (IF ANY)

No related applications.

### STATEMENT OF FEDERALLY SPONSORED RESEARCH OR DEVELOPMENT (IF ANY)

No federally sponsored research or development.

The names of the parties to a joint research agreement if the claimed invention was made as a result of activities within the scope of a joint research agreement.

No joint research agreement.

Reference to a “Sequence Listing,” a table, or a computer program listing appendix submitted on a compact disc and an incorporation by reference of the material on the compact disc. The total number of compact disc including duplicates and the files on each compact disc shall be specified.

N/A.

### BACKGROUND OF THE INVENTION

In dermatology, there are five characteristics to watch for when evaluating the relative threat of moles (nevi). The ABCD (Assymetry, Border, Color, Diameter) have been used for years to educate patients on what to look for in their moles, but a fifth category, Evolution, has recently been added to emphasize that doctors and patients should also be looking for change in those four categories over time. However, there is currently no standard gauge by which dermatologists can evaluate and record any change of color over time. Some dermatologists may take pictures of any questionable nevi for the patient's record, but depending on camera quality and lighting, nevi may appear differently in the picture than they do in reality. To date, dermatologists have relied on asking patients “is that mole darker than last year?” Usually the patient does not know, can't remember, or just guesses. A tool is needed with a standard color scale to accurately chart the relative changes of questionable nevi over time.

### BRIEF SUMMARY OF THE INVENTION

The Nevigraph is a plastic card designed for use in dermatology offices and given to patients to monitor any mole changes over time between dermatologist visits. It includes a millimeter ruler, a range of 10 shades of brown/black with holes to place over the mole while comparing color, a space for the dermatologist to record the mole location, a space for the dermatologist to put their contact information, and a back side that includes descriptions and example pictures illustrating the ABCDEs of dermatology.

### BRIEF DESCRIPTION OF THE SEVERAL VIEWS OF THE DRAWING (IF ANY)

Drawing one (1) shows the front side of the Nevigraph.

Drawing two (2) shows the back side of the Nevigraph.

### DETAILED DESCRIPTION OF THE INVENTION

The Nevigraph is a credit card sized (2.25″×3.25″) plastic card with the following features.

Front side:
1. A millimeter ruler to measure the nevi and record current diameter.
2. A series of 10 shades of brown/black to record current color.
3. Holes within each color box to place over the nevi while comparing colors.
4. A space for the dermatologist to record the location of the questionable nevus.
5. A space for the dermatologist to print their office location and contact information.

Back side:
1. A description and example pictures to illustrate the ABCDE categories.

1. This invention includes the following claims that do not currently exist. A dermatological charting tool consisting of a plastic card; a standard scale of 10 shades of brown to black with holes on each through which to compare and record color on said card; a millimeter ruler for recording diameter on said card; a space to record location on the body on said card; and a reference chart of the ABCDE categories in dermatology on said card.
